(19) United States
(12) Patent Application Publication
(10) Pub. No.: US 2005/0124952 A1
(43) Pub. Date: Jun. 9, 2005

(54) Composite outer covers adapted to attain 3-D configurations

(75) Inventors: Georgia L. Zehner, Larsen, WI (US); Duane G. Uitenbroek, Little Chute, WI (US)

Correspondence Address: KIMBERLY-CLARK WORLDWIDE, INC., 401 NORTH LAKE STREET, NEENAH, WI, 54956, US

(73) Assignee: Kimberly-Clark Worldwide, Inc.

(21) Appl. No.: 10/680,967

(22) Filed: Oct. 7, 2003

(51) Int. Cl. A61F 13/20; A61F 13/15

(52) U.S. Cl. 604/385.01

(57) ABSTRACT

Composite outer covers (30) suitable for incorporation into disposable absorbent articles. Composite outer covers (30) include an extensible outer cover material (32) and a non-tensioned elastic (34). The composite outer covers (30) are adapted to attain three-dimensional (3-D) configurations upon activation.

### BACKGROUND

The present invention relates to composite outer covers. More particularly, the present invention relates to composite outer covers suitable for incorporation into disposable absorbent articles.

Disposable absorbent articles such as diapers, training pants, adult incontinent garments and the like are well known. Various configurations of these disposable absorbent articles have been proposed to help produce and maintain the fit of such articles about the body contours of the wearer.

Many of these conventional disposable absorbent articles have included a plurality of folded pleats in the outer cover. The pleats are arranged to expand open as the disposable absorbent article absorbs liquid.

The external or lower surface of many of such disposable absorbent articles may include a nonwoven fibrous material or a matte-finished film material. In some arrangements, pattern embossments have been formed into the lower surface of the outer cover to provide a decorative pattern.

Still other disposable absorbent articles have incorporated an absorbent composite joined to an outer cover composed of elastomeric materials, such as elastomeric, stretch-bonded-laminate materials. Such materials have included a layer of meltblown elastomeric fibers which has been stretched and sandwiched between facing layers composed of a polypropylene spunbond nonwoven fabric. The meltblown layer has typically been pattern-bonded to the facing layers with thermal bonds, sonic bonds and/or adhesive bonds.

Conventional disposable absorbent articles, such as many of those described above, often fail to provide desired levels of fit and/or ease of manufacture. Consequently, there remains a need for disposable absorbent articles having improvements in such properties.

### SUMMARY

In response to the foregoing need, the present inventors undertook intensive research and development efforts that resulted in the discovery of unique composite outer covers suitable for incorporation into a variety of disposable absorbent articles. One version of the composite outer covers of the present invention includes an extensible, liquid impermeable outer cover material and a non-tensioned elastic. The outer cover material has an upper surface and an opposing lower surface. The non-tensioned elastic is associated with at least a portion of a surface of the outer cover material. Upon activation, at least that portion of the composite outer cover adjacent where the outer cover material and the non-tensioned elastic are associated is adapted to have a retraction capability differential of at least 10% and attain a three-dimensional configuration.

In another version, a disposable absorbent article (having a longitudinal centerline and a lateral centerline) includes a fluid permeable liner, a composite outer cover and an absorbent core. The absorbent core is disposed intermediate the liner and the composite outer cover. In this version, the composite outer cover includes an extensible, liquid impermeable outer cover material and a non-tensioned elastic. The outer cover material has an upper surface and an opposing lower surface. The non-tensioned elastic is associated with at least a portion of a surface of the outer cover material. Upon activation, at least that portion of the composite outer cover adjacent where the outer cover material and the non-tensioned elastic are associated is adapted to have a retraction capability differential of at least 10% and attain a three-dimensional configuration. At least that portion of the outer cover material adjacent where the outer cover material and the non-tensioned elastic are associated is adapted to extend no less than 25%.

### DRAWINGS

The foregoing and other features and aspects of the present invention and the manner of attaining them will become more apparent, and the invention itself will be better understood by reference to the following description, appended claims and accompanying drawings, where:

FIG. 1 illustrates a version of the composite outer cover;

FIGS. 2 through 9 illustrate several configurations of the non-tensioned elastic;

FIG. 10A illustrates an application of the composite outer cover in a pre-activated configuration;

FIG. 10B illustrates the application of FIG. 10A in an activated configuration;

FIG. 11A illustrates an application of the composite outer cover in a pre-activated configuration;

FIG. 11B illustrates the application of FIG. 11A in an activated configuration;

FIG. 12A illustrates an application of the composite outer cover in a pre-activated configuration;

FIG. 12B illustrates the application of FIG. 12A in an activated configuration;

FIG. 13A is an exaggerated illustration of a version of the composite outer cover, in a pre-activated configuration, incorporated into a disposable absorbent article;

FIG. 13B illustrates a cross-sectional view of the disposable absorbent article of FIG. 13A;

FIG. 14A is an exaggerated illustration of a version of the composite outer cover, in an activated configuration, incorporated into a disposable absorbent article;

FIG. 14B illustrates a cross-sectional view of the disposable absorbent article of FIG. 14A;

FIG. 15 representatively illustrates a partially cut-away, bottom plan view of the lower surface of a disposable absorbent article incorporating a version of the composite outer cover; and

FIG. 16 representatively illustrates a partially cut-away, bottom plan view of the lower surface of a disposable absorbent article incorporating a version of the composite outer cover.

### DESCRIPTION

As illustrated in FIG. 1, the composite outer cover (30) of the present invention includes a sheet of extensible, outer cover material (32) and a non-tensioned elastic (34). The outer cover material (32) has a bodyfacing or upper surface (36) and an opposing or lower surface (38). The outer cover material (32) and the non-tensioned elastic (34) are connected or otherwise associated together in an operable manner. As used herein when describing the outer cover material (32) in relation to the non-tensioned elastic (34) and vice versa, the term “associated” encompasses configurations in which the non-tensioned elastic (34) is directly joined to the outer cover material (32), and configurations wherein the non-tensioned elastic (34) is indirectly joined to the outer cover material (32) by affixing the non-tensioned elastic (34) to intermediate members which in turn are affixed to the outer cover material (32). The non-tensioned elastic (34) is connected or otherwise associated with the outer cover material (32) prior to extending the outer cover material (32).

The term “extensible”, as used herein, is intended to refer to members or components that can increase in at least one of their dimensions in the x-y plane. For example, the outer cover material (32)—or portions thereof—is desirably capable of extending no less than 10; alternatively, no less than 15; alternatively, no less than 20; alternatively, no less than 25; alternatively, no less than 30; alternatively, no less than 35; alternatively, no less than 40; alternatively, no less than 45; alternatively, no less than 50; alternatively, no less than 60; alternatively, no less than 70; alternatively, no less than 75; alternatively, no less than 80; alternatively, no less than 90; alternatively, no less than 100; alternatively, no less than 110; alternatively, no less than 120; alternatively, no less than 125; alternatively, no less than 130; alternatively, no less than 140; alternatively, no less than 150; alternatively, no less than 155; alternatively, no less than 160; alternatively, no less than 165; alternatively, no less than 170; alternatively, no less than 175; alternatively, no less than 180; alternatively, no less than 185; alternatively, no less than 190; and finally, alternatively, no less than 195% of its unextended length (and/or its unextended width). In addition, the outer cover material (32)—or portions thereof—is desirably capable of extending no more than 200; alternatively, no more than 195; alternatively, no more than 190; alternatively, no more than 185; alternatively, no more than 180; alternatively, no more than 175; alternatively, no more than 170; alternatively, no more than 165; alternatively, no more than 160; alternatively, no more than 155; alternatively, no more than 150; alternatively, no more than 140; alternatively, no more than 130; alternatively, no more than 125; alternatively, no more than 120; alternatively, no more than 110; alternatively, no more than 100; alternatively, no more than 90; alternatively, no more than 80; alternatively, no more than 75; alternatively, no more than 70; alternatively, no more than 60; alternatively, no more than 50; alternatively, no more than 45; alternatively, no more than 40; alternatively, no more than 35; alternatively, no more than 30; alternatively, no more than 25; alternatively, no more than 20; and finally, alternatively, no more than 15% of its unextended length (and/or its unextended width). Thus, the outer cover material (32)—or portions thereof—may have an extensibility ranging between no less than 10% up to no more than 200%; although the approximate extensibility of the outer cover material (32) may vary according to, inter alia, the general design and intended use of the composite outer cover (30).

In particular aspects, suitable extensible outer cover material (32) can provide an elongation of at least 10; alternatively, at least 20; alternatively, at least 30; or, alternatively, at least 40% when subjected to a tensile force of 30 gmf per inch (per 2.54 cm). The outer cover material (32) can also provide a substantially permanent deformation of at least 10; alternatively, at least 15; alternatively, at least 20; alternatively, at least 25; or, alternatively, at least 30% when subjected to a tensile force of 50 gmf per inch (per 2.54 cm) and then allowed to relax, after removal of the tensile force, for a period of 1 minute. It should be readily appreciated that the described removal of the applied force results in a zero applied tensile stress and a zero applied tensile force.

It should be noted that the elongation, extension or permanent deformation properties of the extensible outer cover material (32) are determined when the outer cover material (32) is dry. Additionally, the percentage of elongation, extension or permanent deformation can be determined in accordance with the following formula:
100*[(L−Lₒ)/(Lₒ)]
where:
   L=elongated length; and
   Lₒ=initial length.

The extensible outer cover material (32) can be composed of various materials and is suitably liquid impermeable. For example, the extensible outer cover material (32) can be composed of a necked fabric, a creped fabric, a crimped fiber fabric, an extendable fiber fabric, a bonded-carded fabric, a micro-pleated fabric, polymer films or the like, as well as combinations thereof. The fabrics may be woven or non-woven materials, such as spunbond fabrics. In a particular embodiment, the extensible outer cover material (32) can be composed of an extensible laminate of two or more layers. For example, the extensible outer cover material (32) may be a necked laminate formed from at least one neckable fabric laminated to at least one extendable film material wherein the necked laminate is extensible in at least one direction. The extensible outer cover material (32) may otherwise be a laminate formed from at least one necked fabric laminated to at least one extendable film material. In such a configuration, the laminate need not be necked. For purposes of the present description, the term “nonwoven web” refers to a web of fibrous material that is formed without the aid of a textile weaving or knitting process. The term “fabrics” is used to refer to woven, knitted and nonwoven fibrous webs. An example of a suitable extensible outer cover material (32) is a 60% necked, polypropylene spunbond having a basis weight of about 1.2 osy.

Examples of suitable manufacturing techniques and suitable necked nonwoven fabric materials are described in U.S. Pat. No. 4,965,122, issued to Mormon (attorney docket number 8,730), the entire disclosure of which is incorporated herein by reference in a manner that is consistent (i.e., not in conflict) herewith.

For purposes of the present description, the term “% necked” or “percent neckdown” refers to a ratio or percentage determined by measuring the difference between the pre-necked dimension and the necked dimension of a neckable material, and then dividing that difference by the pre-necked dimension of the neckable material. The percentage of necking (percent neck) can be determined in accordance with the description in the above-identified U.S. Pat. No. 4,965,122.

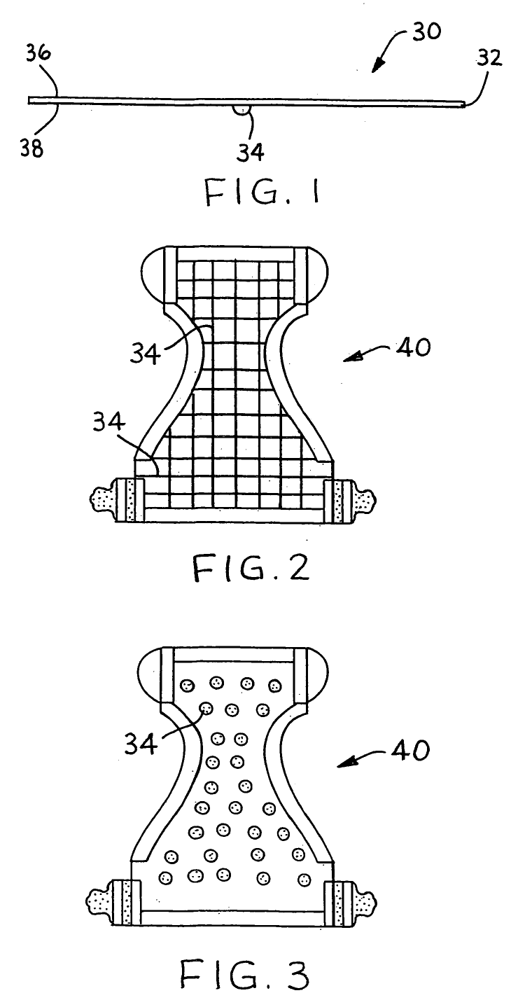

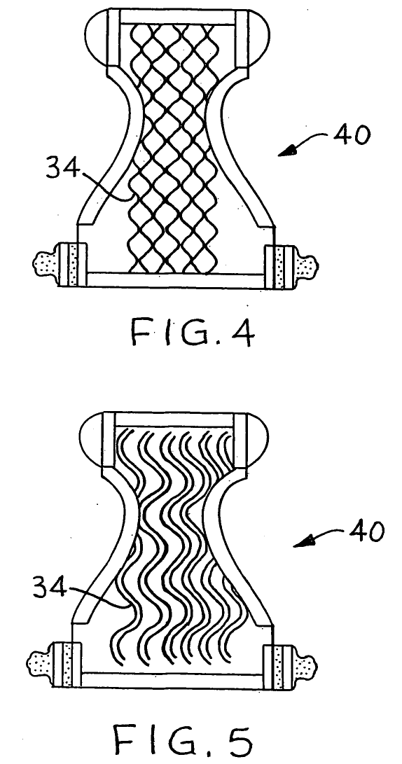

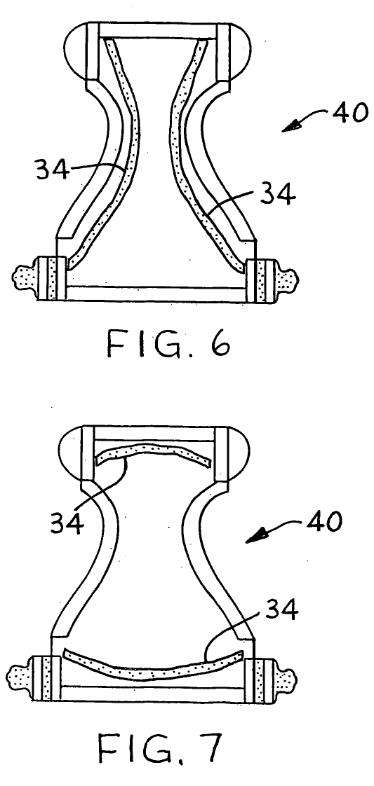

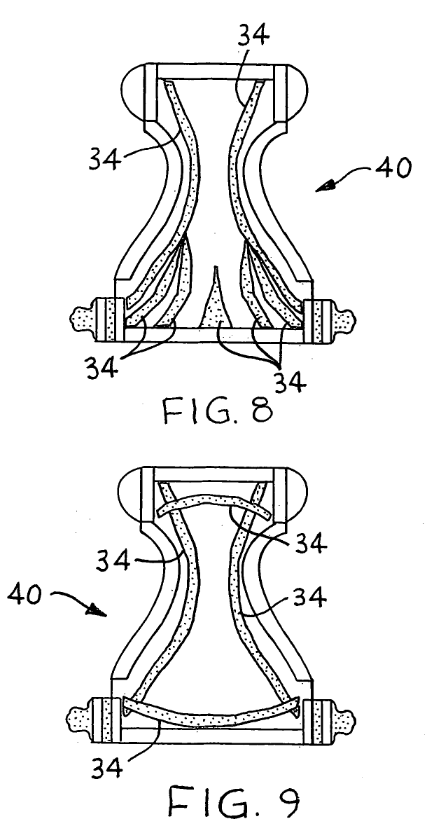

Elastic in the form of strands, bands, ribbons, sheets, laminated composites, films, filaments, fibrous webs, and the like, as well as combinations thereof, are connected or otherwise associated with the extensible outer cover material (32) in an operable manner while the elastic is in the relaxed or non-tensioned condition. The term “elastic”, as used herein, is intended to refer to strands, bands, ribbons, sheets, laminated composites, films, filaments, fibrous webs, and the like, as well as combinations thereof, that have a recovery of at least 25% or more of the extended dimension (e.g., [0.25*(L−Lₒ)]) after being stretched at room temperature. Suitable non-tensioned elastics (34) are generally relatively long and narrow, and are usually applied to the outer cover material (32) so as to be running in a longitudinally-oriented direction, a laterally-oriented direction, and/or a diagonally-oriented direction. Depending on the general design and intended use of the composite outer cover (30), the non-tensioned elastic (34) may be applied to the upper surface (36), the lower surface (38), or both surfaces (36, 38) of the outer cover material (32) in a variety of linear or curvilinear configurations including line, intermittent, dot, dash, and the like. Several non-limiting examples of these configurations are illustrated in FIGS. 2 through 9. The non-tensioned elastic (34) may also be in film, fluid, solid, ribbon, nonwoven, or woven web form in addition to rolls. Several materials suitable to serve as the non-tensioned elastic of the present invention are described in U.S. Pat. No. 6,245,050, issued to Odorzynski et al. (attorney docket number 10,933.1), the entire disclosure of which is hereby incorporated herein by reference in a manner that is consistent (i.e., not in conflict) herewith. For example, identified in U.S. Pat. No. 6,245,050 are certain suitable elastomeric, hot melt, pressure-sensitive adhesives (e.g., Findley H2503 and H2504) available from Bostik Findley, Inc., a business having offices in Wauwatosa, Wis.

The term “vicso-elastic hot melt”, as used herein, is intended to refer to an elastomeric thermoplastic solid that can be melted or extruded at temperatures in excess of 20 to 40° C. The term “pressure sensitive adhesive”, as used herein, is intended to refer to adhesives that bond almost instantaneously when mating surfaces are subjected to pressures forcing them together.

The term “visco-elastic hot melt pressure sensitive adhesive”, “self-adhering composition”, “self-adhering elastic”, and “elastic pressure sensitive adhesive” are used interchangeably herein when referring to elastic materials which adhere to materials suitable for use in disposable absorbent articles and the like, such as paper, cloth, plastic materials, films, filaments, fibers, etc., upon contact or with the use of pressure.

Elastic thermoplastic materials suitable for use in the present invention are desirably soft and flexible. The elastic thermoplastic materials may be supplied to a machine assembly station in roll or bulk form, or they may be extruded through suitable dies. The elastomers may be autogenously bonded to the outer cover material (32) using only heat and pressure, or they may be fusioned or self-bonded to the outer cover material (32) immediately subsequent to their extrusion or they may be co-extruded with a suitable adhesive.

In addition to self-bonding, autogenous bonding, and fusion bonding, the non-tensioned elastic (34) may be connected or otherwise associated with the outer cover material (32) in a variety of configurations via a suitable adhesive. The adhesive may be either of the flexible or rigid type, depending on the manner of application, such as line, intermittent, dot, dash, or any other suitable configuration.

The elastic ribbons may be applied with applicators both hot and cold. They may be extruded and co-extruded whereby they are extruded with one or both of the materials to be bonded to each other. Suitable adhesives include pressure sensitive, cold adhesives, hot melts, releasable adhesives, and pressure sensitive hot melts.

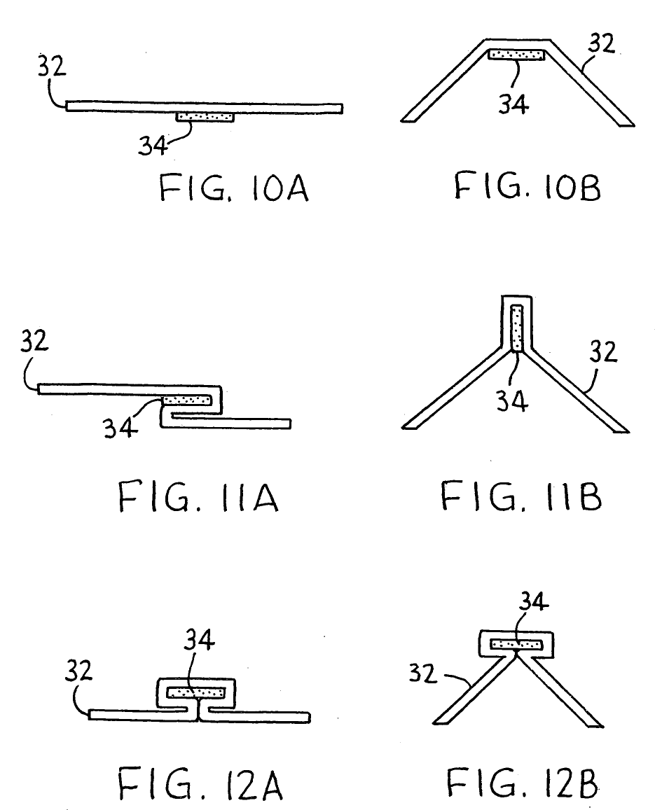

Depending on the general design and intended use of the absorbent article, at least a portion of the non-tensioned elastic (34) may be connected or otherwise associated with at least a portion of the outer cover material (32) in a variety of configurations, including, for example, a flat application (see FIGS. 10A and 10B), a folded application (see FIGS. 11A and 11B), or a “T” folded application (see FIGS. 12A and 12B), as well as combinations thereof.

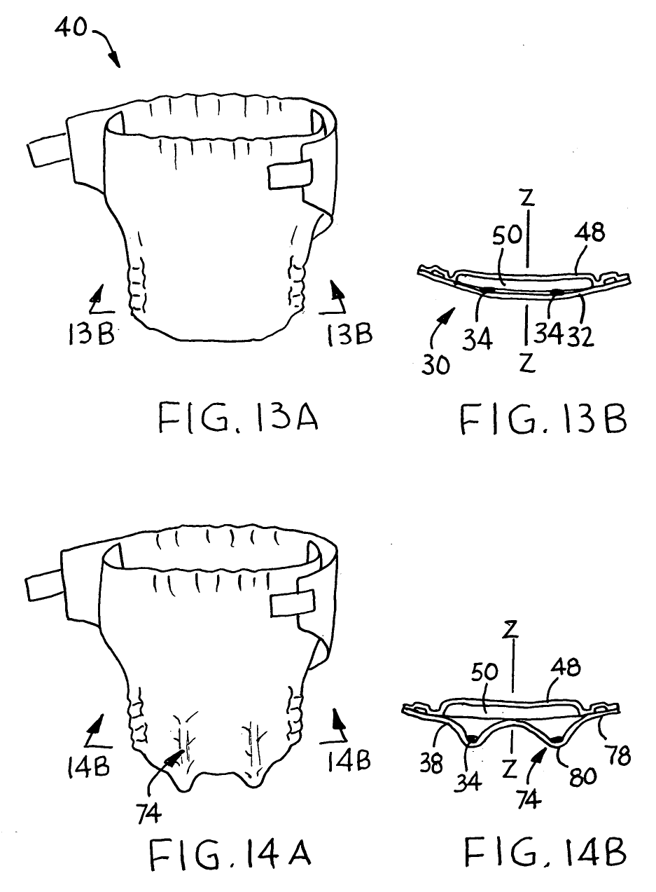

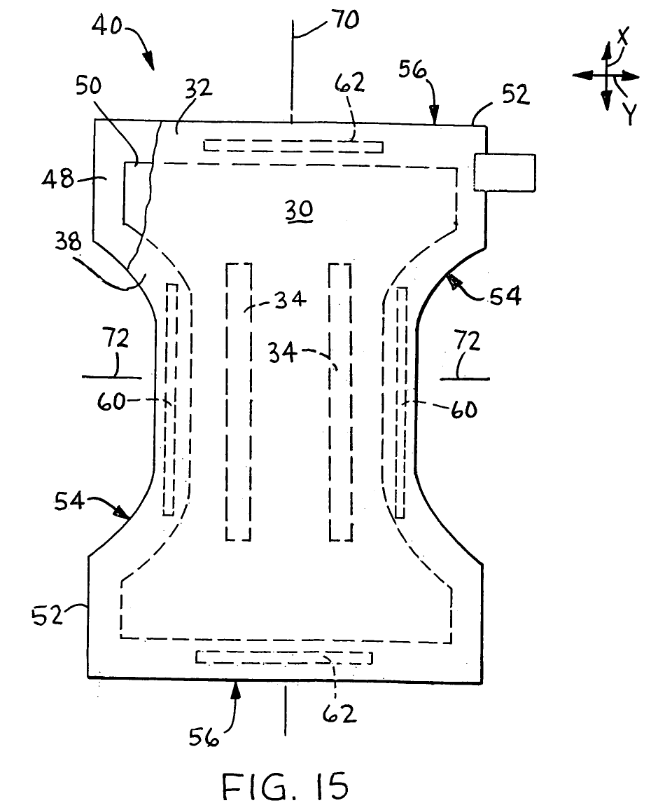

Developers of disposable absorbent articles have long strived to achieve their vision of absorbent articles that are relatively thin, smooth, and flat in their appearance. In the case of absorbent articles such as disposable diapers, training pants, and adult incontinent garments, this vision frequently translates into a desire for absorbent articles that have the look and feel of underwear. Through the discovery of the composite outer covers (30) disclosed herein, this vision is quickly becoming a reality. By associating the extensible outer cover material (32) with the discrete placement of a non-tensioned elastic (34) thereon, the resulting two-dimensional composite outer cover (30) is relatively thin, smooth, and flat as illustrated in FIGS. 13A and 13B. However, when placed into an absorbent article such as a disposable diaper, a portion of the composite outer cover (30) (at least that portion of the composite outer cover (30) adjacent where the outer cover material (32) and the non-tensioned elastic (34) are connected or otherwise associated) may be activated, resulting in at least a portion of the non-tensioned elastic (34) stretching and then retracting back to near its original dimension(s), and at least a corresponding portion of the outer cover material (32) extending and substantially maintaining its extended dimension(s). This phenomenon upon activation (i.e., when the absorbent article is being donned on the wearer or during use by the wearer) creates a three-dimensional (“3-D”) effect on at least one of the surfaces (36, 38) of the outer cover material (32) of the composite outer cover (30). The size, shape, amplitude and frequency of the three-dimensional effect is believed to be determined by the placement of the non-tensioned elastic (34) and the retraction capability differential between the non-tensioned elastic (34) and the corresponding portion(s) of the outer cover material (32).

Use of the term “retraction capability differential” herein is intended to refer to the difference between the amount the non-tensioned elastic (34) retracts from its extended dimension and the amount the extensible outer cover material (32) retracts from its extended dimension. For example, a retraction capability differential of 10% results when a non-tensioned elastic (34) retracts 10% from its extended dimension and the corresponding extensible outer cover material (32) maintains its extended dimension and does not retract. Suitable configurations of the composite outer cover (30)—or portions thereof—typically have a retraction capability differential of no less than 5; alternatively, no less than 10; alternatively, no less than 15; alternatively, no less than 20; alternatively, no less than 25; alternatively, no less than 30; alternatively, no less than 35; alternatively, no less than 40; alternatively, no less than 45; alternatively, no less than 50; alternatively, no less than 55; alternatively, no less than 60; alternatively, no less than 65; alternatively, no less than 70; alternatively, no less than 75; alternatively, no less than 80; alternatively, no less than 85; or finally, alternatively, no less than 90%. In addition, suitable configurations of the composite outer cover (30)—or portions thereof—typically have a retraction capability differential of no more than 95; alternatively, no more than 90; alternatively, no more than 85; alternatively, no more than 80; alternatively, no more than 75; alternatively, no more than 70; alternatively, no more than 65; alternatively, no more than 60; alternatively, no more than 55; alternatively, no more than 50; alternatively, no more than 45; alternatively, no more than 40; alternatively, no more than 35; alternatively, no more than 30; alternatively, no more than 25; alternatively, no more than 20; alternatively, no more than 15; or finally, alternatively, no more than 10%. Thus, suitable configurations of the composite outer cover (30)—or portions thereof—may exhibit a retraction capability differential ranging between no less than 5% up to no more than 95%; although the approximate retraction capability differential may vary according to, inter alia, the general design and intended use of the composite outer cover (30).

The various aspects, benefits, and versions of the composite outer cover (30) will be described in the context of a disposable absorbent article, such as a disposable diaper. It is, however, readily apparent that one or more versions of the present invention could also be employed with other disposable absorbent articles, such as surgical caps and gowns, shoe covers, feminine hygiene articles, children's training pants, adult incontinence garments, and the like. Typically, disposable absorbent articles are intended for limited use and are not intended to be laundered or otherwise cleaned for reuse. A disposable diaper, for example, is discarded after it has become soiled by the wearer. Optionally, a disposable diaper may include a single-use, absorbent insert, and a limited-use outer cover which may be reused several times.

FIGS. 15 and 16 illustrate a disposable diaper (40) as having a front portion (42), a rear portion (44), and a crotch portion (46) located between the front and rear portions. The disposable diaper (40) includes a bodyfacing liner material (48), a composite outer cover (30), and an absorbent core (50) situated between the liner (48) and the composite outer cover (30). The outer edges of the diaper (40) define a periphery (52) with laterally opposed, longitudinally extending side edges (54); longitudinally opposed, laterally extending end edges (56); and a system of elastomeric gathering members, such as a system including leg elastics (60) and waist elastics (62). The longitudinal side edges (54) define leg openings (58) for the diaper (40), and optionally, are curvilinear and contoured. The lateral end edges (56) are illustrated as straight, but optionally, may be curvilinear. The diaper (40) may also include additional components to assist in the acquisition, distribution and storage of bodily waste. For example, the diaper (40) may include a transport layer, such as described in U.S. Pat. No. 4,798,603, issued to Meyer et al. (attorney docket number 8,263), or a surge management layer, such as described in European Patent Application Publication No. 0 539 703 (attorney docket number 9,922), published May 5, 1993.

With regard to the designated surfaces of a disposable absorbent article and its components, the various upper or bodyfacing surfaces are configured to face toward the body of the wearer when the absorbent article is worn by the wearer for ordinary use. The various opposing or lower surfaces are configured to face away from the wearer's body when the disposable absorbent article is worn by the wearer.

The diaper (40) generally defines a longitudinally extending length dimension (64), and a laterally extending width dimension (66), as representatively illustrated in FIG. 16. The diaper may have any desired shape, such as rectangular, I-shaped, a generally hourglass shape, or a T-shape.

The liner (48) and the composite outer cover (30) may be generally coextensive (e.g., FIG. 16), or optionally, may be non-coextensive. Either or both of the liner (48) and the composite outer cover (30) may have length and width dimensions which are generally larger than those of the absorbent core (50) and extend beyond the corresponding dimensions of the absorbent core (50) to provide longitudinal side edges (54) and lateral end edges (56) which may be connected or otherwise associated together in an operable manner. As used herein when describing the composite outer cover (30) in relation to the liner (48) and vice versa, the term “associated” encompasses configurations in which the composite outer cover (30) is directly joined to the liner (48), and configurations where the composite outer cover (30) is indirectly joined to the liner (48) by affixing portions of the composite outer cover (30) to intermediate members which in turn are affixed to at least portions of the liner (48). The composite outer cover (30) and the liner (48) can, for example, be joined to each other in at least a portion of the diaper periphery (52) by attachment mechanisms (not shown) such as adhesive bonds, sonic bonds, thermal bonds, pinning, stitching, or a variety of other attachment techniques known in the art, as well as combinations thereof.

The liner (48) suitably presents a bodyfacing surface which is compliant, soft feeling, and non-irritating to the wearer's skin. Further, the liner (48) may be less hydrophilic than the absorbent core (50), to present a relatively dry surface to the wearer, and is sufficiently porous to be liquid permeable, permitting liquid to readily penetrate through its thickness. A suitable liner (48) may be manufactured from a wide selection of web materials, such as porous foams, reticulated foams, apertured plastic films, natural fibers (for example, polyester or polypropylene fibers), or a combination of natural and synthetic fibers. The liner (48) is suitably employed to help isolate the wearer's skin from liquids held in the absorbent core (50).

Various woven and nonwoven fabrics may be used for the liner (48). For example, the liner (48) may be composed of a meltblown or spunbonded web of polyolefin fibers. The liner (48) may also be a bonded-carded web composed of natural and/or synthetic fibers. The liner (48) may be composed of a substantially hydrophobic material, and the hydrophobic material may, optionally, be treated with a surfactant, or otherwise processed, to impart a desired level of wettability and hydrophilicity. Specifically, the liner (48) may be a nonwoven, spunbond, polypropylene fabric composed of about 2.8 to about 3.2 denier fibers formed into a web having a basis weight of about 22 gsm and a density of about 0.06 g/cc.

The liner (48) may also be surface treated with about 0.3 weight percent of a surfactant mixture that contains a mixture of AHCOVEL Base N-62 surfactant and GLUCOPON 220UP surfactant in about a 3:1 ratio based on a total weight of the surfactant mixture. The AHCOVEL Base N-62 surfactant is purchased from Hodgson Textile Chemicals Inc., a business having offices in Mount Holly, N.C., and includes a blend of hydrogenated ethoxylated castor oil and sorbitan monooleate in a 55:45 weight ratio. The GLUCOPON 220UP surfactant is purchased from Henkel Corporation, Gulph Mills, Pa., and includes alkyl polyglycoside. The surfactant may also include additional ingredients such as aloe. The surfactant may be applied by any conventional means, such as spraying, printing, brush coating, foam or the like. The surfactant may be applied to the entire liner (48) or may be selectively applied to particular sections of the liner (48), such as the medial section along the longitudinal centerline of a diaper, to provide greater wettability of such sections.

The absorbent core (50) may include a matrix of hydrophilic fibers, such as a web of cellulosic fluff, mixed with particles of a high-absorbency material commonly known as superabsorbent material. In a particular version, the absorbent core (50) includes a mixture of superabsorbent hydrogel-forming particles and wood pulp fluff. The wood pulp fluff may be exchanged with synthetic polymeric, meltblown fibers or with a combination of meltblown fibers and natural fibers. The superabsorbent particles may be substantially homogeneously mixed with the hydrophilic fibers or may be non-uniformly mixed.

The absorbent core (50) may have any of a number of shapes. For example, the absorbent core (50) may be rectangular, I-shaped or T-shaped. It is generally desired that the absorbent core (50) be narrower in the crotch portion than the rear or front portion(s).

The high-absorbency material can be selected from natural, synthetic and modified natural polymers and materials. The high-absorbency materials can be inorganic materials, such as silica gels, or organic compounds, such as crosslinked polymers. The term “crosslinked” refers to any means for effectively rendering normally water-soluble materials substantially water insoluble, but swellable. Such means can include, for example, physical entanglement, crystalline domains, covalent bonds, ionic complexes and associations, hydrophilic associations, such as hydrogen bonding, and hydrophobic associations or Van der Waals forces.

Examples of synthetic, polymeric, high-absorbency materials include the alkali metal and ammonium salts of poly(acrylic acid) and poly(methacrylic acid), poly(acrylamides), poly(vinyl ethers), maleic anhydride copolymers with vinyl ethers and alpha-olefins, poly(vinyl pyrolidone), poly(vinyl morpholinone), poly(vinyl alcohol), and mixtures and copolymers thereof. Further polymers suitable for use in the absorbent core include natural and modified natural polymers, such as hydrolyzed acrylonitrile-grafted starch, acrylic acid grafted starch, methyl cellulose, carboxymethyl cellulose, hydroxypropyl cellulose, and the natural gums, such as alginates, xanthum gum, locust bean gum, and the like. Mixtures of natural and wholly or partially synthetic absorbent polymers can also be useful. Processes for preparing synthetic, absorbent gelling polymers are disclosed in U.S. Pat. No. 4,076,663, issued to Masuda et al., and U.S. Pat. No. 4,286,082, issued to Tsubakimoto et al.

The high-absorbency material may be in a variety of geometric forms. It is desired that the high-absorbency material be in the form of discrete particles. However, the high-absorbency material may also be in the form of fibers, flakes, rods, spheres, needles, or the like. Often, the high-absorbency material is present in the absorbent core (50) in an amount of from about 5 to about 100 weight percent based on the total weight of the absorbent core (50).

The composite outer covers (30) of the present invention are suitable for incorporation into a variety of other diaper configurations, as well as training pants, incontinence garments, and other disposable absorbent article configurations. For example, the composite outer covers (30) of the present invention may be incorporated into disposable diapers similar to those described in U.S. Pat. No. 5,509,915, issued to Hanson et al. (attorney docket number 9,922.1), and U.S. Pat. No. 5,192,606, issued to Proxmire et al. (attorney docket number 9,932).

Referring again to FIGS. 15 and 16, illustrated are versions of a diaper (40) in its generally flat-out or pre-activated state. The diaper (40) includes a composite outer cover (30) and a liner (48) which are coextensive and have length and width dimensions generally larger than those of an absorbent core (50). The composite outer cover (30) is associated with and superposed on the liner (48) to thereby form the periphery (52) of the diaper (40). The periphery (52) defines an outer perimeter or edge(s) of the diaper (40). The periphery (52) generally includes longitudinal side edges (54) and lateral end edges (56). The diaper (40) additionally has a longitudinal centerline (70) and a lateral centerline (72). In the illustrated version, the composite outer cover (30) includes an extensible, liquid impermeable outer cover material (32) having a bodyfacing or upper surface (36) and an opposing or lower surface (38). The composite outer cover (30) also includes portions of a non-tensioned elastic (34) connected or otherwise associated with portions of the upper surface (36) of the outer cover material (32). The non-tensioned elastic (34) is positioned inboard from the longitudinal side edges (54) of the diaper (40) and runs in a direction generally parallel to the longitudinally extending length dimension (64) of the diaper (40). The term “inboard” is intended to refer to the direction from an edge (54, 56) toward a respective centerline (70, 72). The term “outboard” is intended to refer to a direction away from a respective centerline (70, 72). As a result of the illustrated diaper (40) being in its pre-activated state, the diaper (40) thus retains its somewhat two-dimensional configuration with the lower surface (38) of the composite outer cover (30) being substantially smooth in appearance.

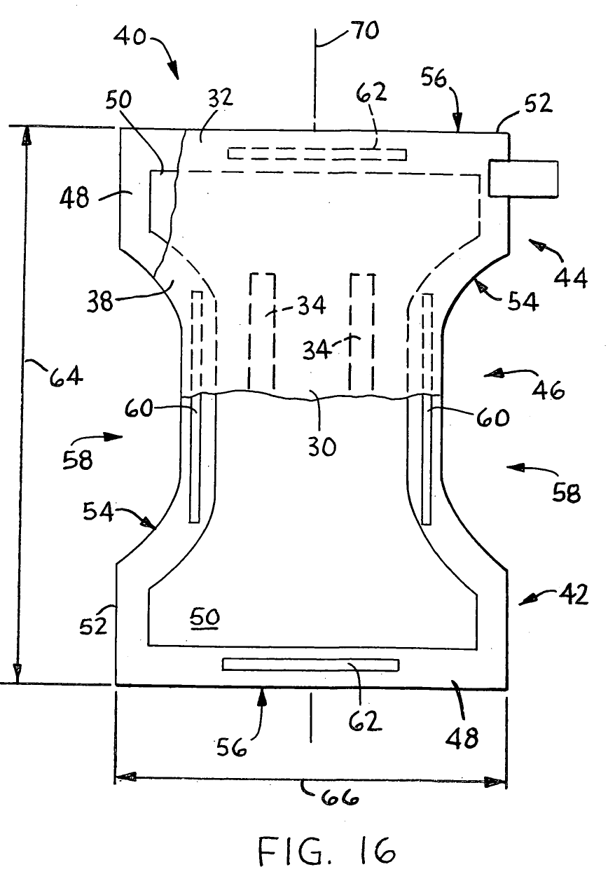

Turning now to FIGS. 14A and 14B, upon donning on the wearer or while in use by the wearer, at least a portion of the composite outer cover (30) of the diaper (40) is activated. That portion of the composite outer cover (30) that is activated typically has a retraction capability differential of at least 10% and is adapted to attain a three-dimensional configuration. In the illustrated version, at least that portion of the composite outer cover (30) adjacent where the outer cover material (32) and the non-tensioned elastic (34) are connected or otherwise associated attains the three-dimensional configuration by forming a spacer element (74). As illustrated in FIG. 14A, the spacer element (74) runs in a longitudinally-oriented direction and is disposed inboard from a longitudinal side edge (54) toward the longitudinal centerline (70).

In another version (not specifically illustrated), at least a portion of the composite outer cover (30) of the diaper (40) is activated upon donning on the wearer or while in use by the wearer. That portion of the composite outer cover (30) that is activated typically has a retraction capability differential of at least 10% and is adapted to attain a three-dimensional configuration. In this version, at least that portion of the composite outer cover (30) adjacent where the outer cover material (32) and the non-tensioned elastic (34) are connected or otherwise associated attains the three-dimensional configuration by forming a spacer element (74). In such a version, the spacer element (74) runs in a laterally-oriented direction and is disposed inboard from the lateral end edge (56) toward the lateral centerline (72) (see, for example, FIG. 7).

Although adapted to attain three-dimensional configurations such as the previously described longitudinally- and/or laterally-oriented spacer element(s) (74) upon activation, the composite outer covers (30) disclosed herein are also capable of obtaining a variety of other spacer element (74) configurations upon activation. Several such configurations are illustrated in FIGS. 2 through 9.

As illustrated in FIG. 14B, a spacer element (74) typically has at least a base region (78) and a distal edge (80). The base region (78) and the distal edge (80) are in spaced relation to each other and define the height (typically in the z-direction) of the spacer element (74). The base region (78) and the distal edge (80) may be in a substantially parallel, non-parallel, rectilinear or curvilinear relationship. In addition, the spacer element (74) may have a variety of different cross-sectional areas including circular, square, rectangular or any other suitable shape. Suitably, a spacer element (74) has a height in accordance with its general design and intended use. In certain versions of the present invention, suitable spacer elements (74) have a height of at least 5, or, alternatively, at least 10 mm to no more than 25 mm.

Although described herein as being substantially entirely extensible, one of skill in the art will readily appreciate that certain portions of the composite outer cover (30) can be made substantially non-extensible by affixing one or more portions of the extensible outer cover material (32) to one or more portions of a substantially non-extensible component, such as, for example, a substantially non-extensible liner (48) or a substantially non-extensible absorbent core (50).

As noted above, conventional disposable absorbent articles such as diapers typically include an absorbent core. Generally when an absorbent core absorbs liquid, it increases in volume. Unless the outer cover is allowed to accommodate the expanding absorbent core, the maximum capacity of the absorbent core oftentimes cannot be achieved. One approach to accommodate an expanding absorbent core has been to provide a plurality of pleats in the outer cover material. These pleats serve as a mechanism for allowing the absorbent core to expand out and away from the wearer's body. Although often effective for its intended purpose, a pleated outer cover typically uses more outer cover material and is more complex to process. Consequently, the cost of a pleated outer cover is generally greater than the cost of an otherwise similar outer cover that is not pleated. Another approach to accommodate an expanding absorbent core has provided for the utilization of outer cover materials that extend in one or more directions. Once extended, however, these extensible outer cover materials are not able to retract back to near the original dimension. Consequently, when the absorbent core is compressed after having increased its volume the outer cover adjacent the compressed area oftentimes blouses and provides a disposable absorbent article that is less aesthetically pleasing in appearance. The composite outer covers (30) of the present invention contribute to a reduction in these problems and will allow developers of disposable absorbent articles to take a step toward achieving their vision of disposable absorbent articles that are relatively thin, smooth and flat in their appearance. It is believed that the composite outer covers (30) discussed herein also represent a contribution toward the development of disposable absorbent articles that are relatively easier to manufacture and present and maintain an outer cover that is more aesthetically pleasing in appearance. Specifically, when the absorbent core of a disposable absorbent article incorporating the present invention absorbs liquid and increases in volume, the resultant expansion of the absorbent core exerts a tensile stress and/or force on the composite outer cover (30). The stress and/or force applied on the composite outer cover (30) by the expanding absorbent core may also serve to activate the composite outer cover (30).

### Test Method(s)

A suitable technique for determining the amount of elongation and/or retractive force parameters of a selected component or material can employ ASTM Standard Test Method D882 (Tensile Method for Tensile Properties of Thin Plastic Sheeting) dated December 1995, with the following particulars. The “width” of the test sample will be a cross-wise width which can be conveniently obtained from the product being tested, and is desirably about 2 inches (about 5 cm). The test sample width is perpendicular to the direction of the tensile force applied during the testing. With regard to the illustrated configurations, for example, the test sample “width” generally corresponds to the length-wise dimension of the extensible outer cover material (32), for example, along the longitudinally-extending length dimension of the article. The initial separation of the jaws of the tensile tester is 3 inches (7.62 cm), and the moving jaw is moved at a constant rate of 50 mm/min. The moving jaw is stopped at an extension of 50 mm for a period of 10 seconds, and then returned back to its initial starting position at a rate of 50 mm/min. The force-extension curve to the complete tension and retraction cycle can be recorded on a conventional computer equipped with commercially available software, such as TestWorks for Windows, version 3.09, which is available from MTS System Corporation, a business having a location at 14000 Technology Drive, Eden Prairie, Minn. The obtained data is normalized and reported in appropriate units of force per unit length of sample “width” (e.g., grams-force per inch or Newtons per inch; or grams-force per centimeter or Newtons per centimeter).

Having described the invention in rather full detail, it will be readily apparent that various changes and modifications can be made without departing from the spirit of the invention. All of such changes and modifications are contemplated as being within the scope of the invention as defined by the appended claims and any equivalents thereto.

1. A disposable absorbent article having a longitudinal centerline (70) and a lateral centerline (72), the article comprising:
a fluid permeable liner (48);
a composite outer cover (30), the composite outer cover (30) including: (a) an extensible, liquid impermeable outer cover material (32) having an upper surface (36) and an opposing lower surface (38); and (b) an non-tensioned elastic (34), the non-tensioned elastic (34) being associated with at least a portion of a surface (36, 38) of the outer cover material (32), wherein upon activation at least a portion of the composite outer cover (30) adjacent where the outer cover material (32) and the non-tensioned elastic (34) are associated is adapted to (i) have a retraction capability differential of at least 10% and (ii) attain a three-dimensional configuration, and wherein at least that portion of the outer cover material (32) adjacent where the outer cover material (32) and the non-tensioned elastic (34) are associated is adapted to extend no less than 25%; and
an absorbent core (50) disposed intermediate the liner (48) and the composite outer cover (30).

2. The disposable absorbent article of claim 1, wherein the three-dimensional configuration is a spacer element (74).

3. The disposable absorbent article of claim 2, wherein the composite outer cover (30) is associated with and superposed on the liner (48) to thereby form a periphery (52), the periphery (52) generally including longitudinal side edges (54) and lateral end edges (56).

4. The disposable absorbent article of claim 3, wherein at least a portion of the spacer element (74) is disposed inboard from a longitudinal side edge (54) toward the longitudinal centerline (70).

5. The disposable absorbent article of claim 4, wherein the non-tensioned elastic (34) is associated with the upper surface (36) of the outer cover material (32).

6. The disposable absorbent article of claim 3, wherein at least a portion of the spacer element (74) is disposed inboard from a lateral end edge (56) toward the lateral centerline (72).

7. The disposable absorbent article of claim 6, wherein the non-tensioned elastic (34) is associated with the upper surface (36) of the outer cover material (32).

8. The disposable absorbent article of claim 3, wherein at least a portion of the spacer element (74) is disposed outboard from the longitudinal centerline (70).

9. The disposable absorbent article of claim 8, wherein at least a portion of the spacer element (74) resides on the longitudinal centerline (70).

10. The disposable absorbent article of claim 9, wherein the non-tensioned elastic (34) is associated with the upper surface (36) of the outer cover material (32).

11. The disposable absorbent article of claim 3, wherein at least a portion of the spacer element (74) is disposed outboard from the lateral centerline (72).

12. The disposable absorbent article of claim 11, wherein at least a portion of the spacer element (74) resides on the lateral centerline (72).

13. The disposable absorbent article of claim 12, wherein the non-tensioned elastic (34) is associated with the upper surface (36) of the outer cover material (32).

14. A composite outer cover (30) suitable for incorporation into a disposable absorbent article, the composite outer cover (30) comprising: (a) an extensible, liquid impermeable outer cover material (32) having an upper surface (36) and an opposing lower surface (38); and (b) a non-tensioned elastic (34), the non-tensioned elastic (34) being associated with at least a portion of a surface (36, 38) of the outer cover material (32), and wherein upon activation at least that portion of the composite outer cover (30) adjacent where the outer cover material (32) and the non-tensioned elastic (34) are associated is adapted to: (i) have a retraction capability differential of at least 10% and (ii) attain a three-dimensional configuration.

15. The composite outer cover (30) of claim 14, wherein the three-dimensional configuration has a distal edge (80) and a base region (78), the distal edge (80) and the base region (78) being in spaced relation from each other.

16. The composite outer cover (30) of the claim 15, further comprising a longitudinal centerline (70) and a lateral centerline (72).

17. The composite outer cover (30) of claim 16, wherein the three dimensional configuration is a spacer element (74).

18. The composite outer cover (30) of claim 17, wherein at least a portion of the spacer element (74) is disposed outboard from the longitudinal centerline (70).

19. The composite outer cover (30) of claim 18, wherein at least a portion of the spacer element (74) resides on the longitudinal centerline (70).

20. The composite outer cover (30) of claim 19, wherein the non-tensioned elastic (34) is associated with the upper surface (36) of the outer cover material (32).

21. The composite outer cover (30) of claim 17, wherein at least a portion of the spacer element (74) is disposed outboard from the lateral centerline (72).

22. The composite outer cover (30) of claim 21, wherein at least a portion of the spacer element (74) resides on the lateral centerline (72).

23. The composite outer cover (30) of claim 22, wherein the non-tensioned elastic (34) is associated with the upper surface (36) of the outer cover material (32).
